(19) United States
(12) Patent Application Publication
(10) Pub. No.: US 2013/0274575 A1
(43) Pub. Date: Oct. 17, 2013

(54) CARBON DIOXIDE GAS MEASUREMENT SYSTEM AND METHOD

(71) Applicant: EXOSTAT MEDICAL, INC., Prior Lake, MN (US)

(72) Inventors: Kent R. Winger, Prior Lake, MN (US); Irvin Pierskalla, Prior Lake, MN (US); Mark B. Winger, Ramsey, MN (US)

(73) Assignee: EXOSTAT MEDICAL, INC., Prior Lake, MN (US)

(21) Appl. No.: 13/796,853

(22) Filed: Mar. 12, 2013

Related U.S. Application Data
(60) Provisional application No. 61/622,892, filed on Apr. 11, 2012.

(51) Int. Cl. A61B 5/145; A61B 5/1477

(52) U.S. Cl. CPC A61B 5/14542; A61B 5/1477
USPC 600/353

(57) ABSTRACT

A measurement system for measuring carbon dioxide in a tissue sample is provided. The system includes a housing having a distal end and a proximal end, the distal end including a recessed area; electrodes positioned within the housing, said electrodes in mating relationship; a conductive media in contact with said electrodes; and a recognition layer disposed between said electrodes and said recessed area.

### FIELD OF THE INVENTION

The present invention relates generally to systems and methods for measuring a physiological parameter in a patient. More particularly, the invention relates to measuring the partial pressure of CO.sub.2 in a mucosal tissue of a patient to determine the degree of perfusion failure.

### BACKGROUND OF THE INVENTION

There is a continuing need for improved devices and methods for determining one or more physiologic parameters of a patient. Often, such physiologic parameters are determined by detecting or measuring the quantity of concentration of an analyte associated with the physiologic parameter within a tissue of a patient. Noninvasive techniques for analyte detection are preferred over invasive detection techniques because invasive procedures result in stress and discomfort to patients. For example, conventional detection of blood analytes often involves drawing a sample of blood from the patient and subjecting the sample to in vitro testing for a specific analyte. This technique suffers from a number of drawbacks. First, drawing blood requires the puncturing of a patient's skin and creates a risk of infection. Second, hypodermic needles used in drawing blood may also pose a risk of accidental infection to health care professionals such as phlebotomists who routinely use the needles and sanitary workers who handle contaminated needles. Third, the technique of drawing blood and in vitro testing is not easily adaptable for real-time and continuous monitoring of changes in analyte.

Nevertheless, analyte detection is of course an essential process in medical diagnosis. For example, very low blood flow, or low “systemic perfusion,” occurs typically because of low aortic pressure and can be caused by a number of factors, including hemorrhage, sepsis, and cardiac arrest. The body responds to such stress by reducing blood flow to the gastrointestinal tract to spare blood for other, more critical organs. Thus, when blood flow from the heart is reduced, blood flow is generally maintained to critical organs, such as the brain, which will not survive long without a continuous supply of blood, while blood flow is restricted to less critical organs, whose survival is not as threatened by a temporary reduction in blood flow. For example, blood flow to the stomach, intestines, esophagus and oral/nasal cavity is drastically reduced when there is a reduced blood flow from the heart or when a patient is experiencing circulatory shock. For this reason, decreased blood flow to the splanchnic blood vessels provides an indication of perfusion failure in a patient. Physicians commonly take advantage of this phenomenon by taking CO.sub.2 and pH measurements in the stomach and intestine to assess perfusion failure.

Assessment of CO.sub.2 concentration in the less critical organs, i.e., those organs to which blood flow is reduced during perfusion failure, has also been useful in perfusion assessment. Carbon dioxide production, which is associated with metabolism, continues in tissues even during conditions of low blood flow. The concentration of CO.sub.2 builds up in tissues experiencing low blood flow because CO.sub.2 is not rapidly carried away. Correspondingly, O.sub.2 is consumed as CO.sub.2 is generated. This CO.sub.2 build-up (an increase in partial pressure of CO.sub.2 (pCO.sub.2)) in the less critical organs in turn results in a decrease in pH. Therefore, perfusion failure is commonly assessed by measuring pH or pCO.sub.2 at these sites, especially in the stomach and intestines. For examples of catheters used to assess pH or pCO.sub.2 in the stomach or intestines, see, e.g., U.S. Pat. Nos. 3,905,889; 4,016,863; 4,632,119; 4,643,192; 4,981,470; 5,105,812; 5,117,827; 5,174,290; 5,341,803; 5,411,022; 5,423,320; 5,456,251; and 5,788,631.

A number other patents discuss the measurement tissue analytes. For example, U.S. Pat. No. 5,579.763 to Weil et al. discloses the introduction of a catheter with a carbon dioxide sensor through the nasal or oral passage into the esophagus. While CO.sub.2 measurements in the esophagus involve only moderate invasiveness, it is unnecessarily invasive to the body since it involves moving a catheter down past the epiglottis to reach the esophagus. It would be desirable if an even less invasive method were available to measure perfusion failure and to indicate the state of the patient as a result of perfusion failure and as the result of blood infusion or other methods taken to increase perfusion.

Similarly, U.S. Pat. No. 6,055,447 to Weil also relates to carbon dioxide measurements and describes that the sensor may be placed against mucosal surface in the mouth or nose other than the sublingual area. U.S. Pat. No. 6,216,024 to Weil et al. discusses a device for assessing perfusion failure that comprises a carbon dioxide sensor for lying against a mucosal surface of the upper digestive/respiratory tract of a patient, an isolating means for inhibiting air flow around the mucosal surface, and indicating means operatively connected to the sensor for indicating a degree of perfusion failure of the patient.

U.S. Pat. No. 8,369,920 to Castillo et al. discloses a sensor that lies against a mucosal surface. The sensor includes a seal that extends 360 degrees around the sensor end that presses against the mucosal surface leaving the sensor exposed to contact the tissue. However, the sensor arrangement of Castillo is untested and the cell design may affect measured conductance adversely.

Thus what is needed is a measurement system for measuring a physiological parameter of a patient such as CO.sub.2 that can be used in a variety of mucosal tissues, is non-invasive and creates a microenvironment for measurement.

### BRIEF SUMMARY OF THE INVENTION

The foregoing problems of the prior art are addressed with the measurement system in accordance with the invention.

In accordance with one aspect of the present invention, a carbon dioxide measurement system is provided for assessing the non-invasive measurement of CO.sub.2. The carbon dioxide measurement system includes a probe that can be placed sublingually or against any mucosal surface of a patient's body.

In one aspect of the invention, the device may comprise a hand-held device with a reusable probe or preferably a disposable probe used to measure the level of carbon dioxide sublingually—a tissue bed that is extremely sensitive to subtle and early changes in the body that are a prelude to catastrophic collapse of pressure (shock) and ultimately, failure of organ function.

In another aspect of the invention, the device delivers immediate, quantitative information to physicians for patient assessment.

In another aspect of the carbon dioxide measurement system in accordance with the invention, the system utilizes an electrode conductance sensor positioned within a housing which creates a microenvironment to capture and measure gas phase carbon dioxide through an ionic exchange within the micro environment.

The carbon dioxide measurement system in accordance with the invention delivers both early detection and a sensitive treatment guide in order to rapidly identify and optimize therapies used to reverse compromised perfusion in emergency or clinic settings.

Other physiologic measurements (blood pressure, temperature, heart rate) and blood tests frequently fall short of providing the clinician with the information required to both detect hemodynamic collapse before it becomes obvious and manage therapy to reverse it.

These other features of the invention will be best understood from the following description when read in conjunction with the accompanying drawings.

### BRIEF DESCRIPTION OF THE DRAWINGS

For a better understanding of the invention, and to show how the same may be carried into effect, reference will now be made, by way of example, to the accompanying drawings, in which:

FIG. 1 is an illustration of a basic electrode component of the carbon dioxide measurement system in accordance with the invention.

FIG. 2 is an exemplary illustration broadly depicting the schematics of the processor of the carbon dioxide measurement system in accordance with the invention.

FIG. 3 is a schematic view of an exemplary embodiment of an conductivity electrode component of the carbon dioxide measurement system in accordance with the invention.

FIG. 4 is a perspective view of the conductivity electrode in accordance with the invention.

FIG. 5 is a graph illustrating the effect on conductance resulting from the size of the gap between the electrodes.

FIG. 6 is a graph illustrating the effect on conductance resulting from the size of electrode plates.

### DETAILED DESCRIPTION OF THE INVENTION

The conductivity of a solution is a measure of its ability to conduct electricity. Conductivity is traditionally determined by measuring the impedance (AC resistance) of the Solution between two electrodes. The impedance is the measure of opposition that a circuit presents to the passage of an electric current when a voltage is applied. In quantitative terms, it is the complex ratio of the voltage to the current in an alternating current (AC) circuit. The inverse to impedance is electrical conductance, the ease at which an electric current passes. The sensor in accordance with the invention is a conductivity sensor.

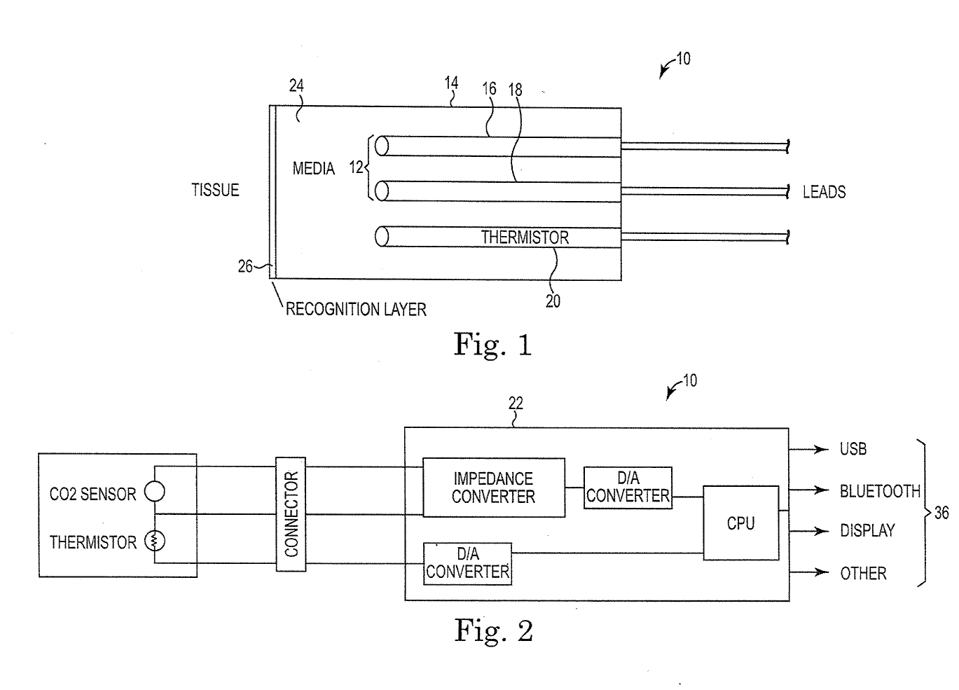

Referring generally to FIGS. 1 and 2, the carbon dioxide measurement system 10 in accordance with the invention utilizes an conductivity sensor 12 positioned within a housing 14. The conductivity sensor 12 includes electrodes 16 and 18. The sensor assembly may optionally include a temperature measurement device 20 to be used to adjust the conductance shift created by temperature changes. The conductivity and temperature measurements taken within the measurement system 10 are communicated to a microprocessor 22 which utilizes an algorithm to correct for the influence of temperature, and calculate a CO.sub.2 reading displayed in units of partial pressure presentable on multiple display formats 36. These CO.sub.2 measurements can be taken in virtually all locations in tissue in living beings as well as industrial uses where minute CO.sub.2 level changes need measurement and or tracking. This device can perform both single point measurements as well as continuous CO.sub.2 monitoring.

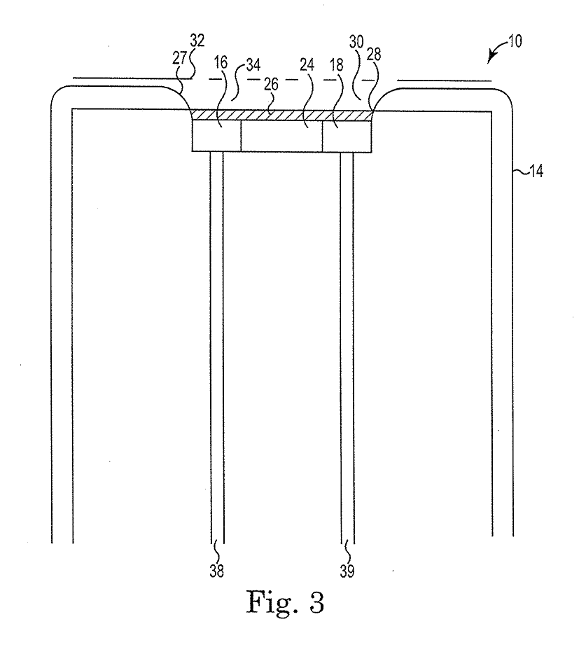

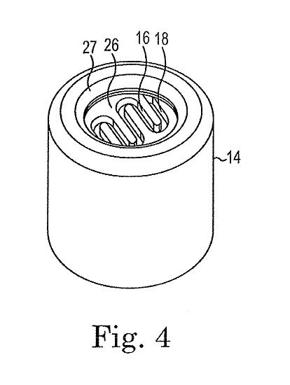

Referring now to FIGS. 3 and 4 the conductivity sensor 12 in accordance with the invention broadly includes housing 14, electrodes 16 and 18. Electrodes 16 and 18 are disposed within a conductive media 24 and connected by leads 38, 39 to microprocessor 22. Electrodes 16, 18 are covered by a membrane or recognition layer 26. Because conductivity is not specific to the ion of interest, the recognition layer 26 is employed to isolate the species of interest. In this case the species of interest is CO2, which forms carbonic acid in the cell that in turn dissociates to hydrogen (H+) and bicarbonate (HCO3−) ions.

Recognition layer 26 may comprise a fluoropolymer, polydimethylsiloxane, hydrogel, or any plastic or elastomeric material. Recognition layer 26 may also comprise a microporous membrane. The recognition layer 26 may be a monolithic structure or a composite structure. In one embodiment of the composite structure, a microporous membrane may be attached to one or both sides of the recognition layer 26.

Recognition layer 26 overlays the media 24, electrodes 16, 18 assembly. Alternatively, those of skill in the art will appreciate that while housing 14 is depicted as having a circular shape other shapes may include square shaped, rectangular shaped and other shapes known to those of skill in the art. In such a case, the recognition layer 26 will take a similar form. The recognition layer 26 allows CO.sub.2 as its primary diffusing gas but may also be permeable to other gases known to those of skill in the art.

As best seen in FIGS. 3 and 4, housing forms an isolation chamber 30 circumferentially surrounded by housing sides 27, 28 and on an inner side by recognition layer 26. When the sensor assembly is placed on patient mucosal tissue 32 the configuration forms a microenvironment 34 that allows the tissue pCO.sub.2 to equilibrate therewithin.

There is not any conductance between the electrodes unless introduced by the conductive media 24. The media 24 may be an ultra-pure media consisting of any number of components, some of which may act as a buffer agent and/or conductance enhancer. The media may comprise carbon nanotubes, enzymes, hydrogels and other conductance enhancers known to those of skill in the art. The media 24 may be encapsulated between the electrode assembly or alternatively the electrodes may be completely surrounded by or embedded in the media, as best seen in FIG. 1. The media electrical conductance will increase with the increased presence of CO.sub.2 as well as decrease with a decreased level of CO.sub.2.

The electrodes 16, 18 also include leads 38, 39 which transfer current between the electrodes and the processor 22. Processor 22 then calculates impedance and conductance based on the measured current.

The device in accordance with the invention may also include a temperature measurement device 20, as best seen in FIG. 1, for measuring temperature within the housing 14. Temperature alters the conductance of media so therefore must be measured and conductance adjusted prior to calculating CO.sub.2 levels in the fluid.

As note previously, the shape of the carbon dioxide measurement device can vary depending on the location CO.sub.2 is to be measured. A round tipped sensor may be required for soft tissue measurements or in a fluid setting while a flat tipped probe may be required for harder surfaces or permanent placement.

The measurements taken by the electrodes may be transferred to the processor via hardwire or wireless transmission.

The processor, as best seen in FIG. 2, may contain software including algorithms to correct the CO.sub.2 by evaluating the temperature, convert the electrical measurement to partial pressure, and track and trend measurement changes. The processor may also include a timer or clock feature for tracking and reporting purposes. The processor may include input capabilities to allow manual and electronic data input such as test subject, time, and location. The processor communicates the completed data to a variety of displays such as an integrated screen, computer/laptop, smart phones, remote servers and the like.

In operation, an analyzer (not shown) applies an alternating voltage to the electrodes. The resultant electric field causes the ions to move back and forth producing a current. This ionic current depends on the total concentration of ions in solution and on the length and area of the solution through which the current flows.

The cell design impacts the measured conductance of a known concentration. The current path is defined by the sensor geometry, or cell constant, which has units of 1/cm (distance/area). Multiplying the measured conductance by the cell constant corrects for the effect of sensor geometry and normalizes the results, which is an important factor when reporting results in units of conductivity (S/cm).

The concept of a cell constant may be leveraged to optimize ceil design for the CO2 range of interest. Those of skill in the art will appreciate that a smaller cell constant relates to a larger magnitude conductance signal for a given concentration. The larger magnitude signals allow for finer resolution. A resolution of 1 mmHg is acceptable for the reporting of sublingual pCO2 and a resolution of 0.1 mmHg is preferable.

The cell constant has a geometric interpretation of the distance D or the “gap” between the electrodes divided by the area A of the electrodes. Thus, the shorter the distance between the electrodes, the smaller the cell constant. In the same manner the larger the area of the electrodes, the smaller the cell constant. The size of the electrode must be balanced with the need for shorter response times. To optimize these somewhat conflicting needs, the area of the electrode is maximized while minimizing the resultant volume. This size can be modified to fit the size restrictions of the various measurement locations.

In another aspect of the sensor, to maximize the cell length within the limited footprint of the sensor, the electrodes may be a serpentine, zig-zag, or similar cell shape in which the two electrodes are in mating relationship separated by a substantially equal gap therebetween. Thus, as best seen in FIG. 4, the serpentine shape of the two electrodes creates a mating relationship with a substantially equal “gap” between the two electrodes. The electrodes 16, 18 are preferably platinum or platinum coated but those of skill in the art will appreciate that electrodes 16, 18 may also comprise gold, carbon, stainless steel, or any conductive material demonstrating good electrochemical inertness.

In normal practice, cell constants are determined indirectly by measuring a solution of Known conductivity. The cell constant is the ratio of the known conductivity (μS/cm) to the measured conductance (μS).

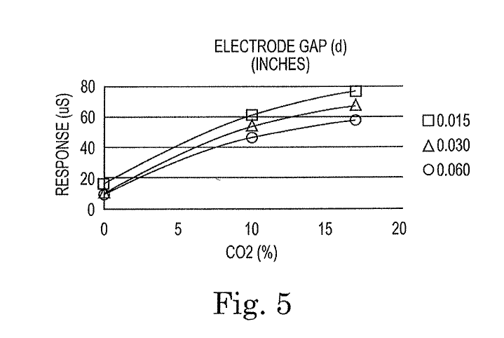

Referring now to Table I and FIG. 5 an experiment was conducted to demonstrate the importance and effect of electrode gap D on conductance. Metal filaments were fixed to opposing plates that were separated by an adjustable distance. Conductance measurements were made in aqueous solutions tonometered with mixed CO2/N2 gases at a controlled temperature. The data set forth in Table I was plotted on the graph in FIG. 5. As can be seen, a smaller electrode gap (d) provides greater conductance measurements. The gap is preferably from 0.005 to 0.030 inches and more preferably 0.020 inches.

TABLE I
Conductance of CO2 Tonometered Solutions

| | Electrode Gap (inches) | | |
|---|---|---|---|
| % CO2 | 0.06 | 0.03 | 0.015 |
| 0 | 10 | 10 | 16 |
| 10 | 46 | 53 | 60 |
| 17 | 57 | 67 | 76 |

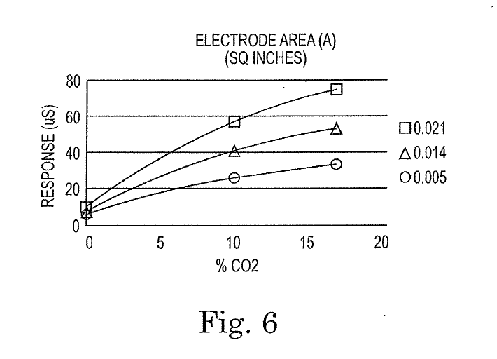

Referring to Table II below and FIG. 6 a second experiment was conducted to demonstrate the effect of electrode size (A) on conductance measurements. In this experiment, measurements were made with an electrode of known size. Subsequently portions of the electrode were removed and additional measurements made. Conductance measurements were made in aqueous solutions tonometered with mixed CO2/N2 gases at a controlled temperature and then plotted on the graph in FIG. 6. As can be seen in FIG. 6 a larger electrode size (A) provides larger dynamic range conductance measurements.

TABLE II
Conductance of CO2 Tonometered Solutions

| | Electrode Area (sq. in.) | | |
|---|---|---|---|
| % CO2 | 0.005 | 0.014 | 0.021 |
| 0 | 7 | 9 | 10 |
| 10 | 26 | 41 | 57 |
| 17 | 33 | 53 | 75 |

Those of ordinary skill in the art will appreciate that the novel carbon dioxide measurement system in accordance with the invention may be used in numerous care settings where sudden blood loss, cardiac arrest, severe infection or rapid fluid changes occur including but not limited to emergency care during transport as well as once the patient arrives at the hospital: military emergency care units; intensive care unit; post-operative recovery units: dialysis centers; and, long-term nursing care facilities.

The various components of the carbon dioxide measurement system disclosed herein may be embodied exclusively as or in combination with a method, device, or computer program product. Accordingly, the various aspects of the present invention may be embodied in any combination of hardware or software aspects. Furthermore, the presently described invention may include a computer program embodied in a non-transitory, tangible medium of expression having computer usable program code embodied in the medium. Although various representative embodiments of this invention have been described above with a certain degree of particularity, those of ordinary skill in the art will appreciate that numerous alterations may be made to the disclosed embodiments without departing from the spirit or scope of the inventive subject matter set forth herein.

We claim:

1. A measurement system for measuring carbon dioxide in a tissue sample comprising:
a housing having a distal end and a proximal end, said distal end including a recessed area;
electrodes positioned within said housing, said electrodes in mating relationship;
a conductive media in contact with said electrodes; and
a recognition layer disposed between said electrodes and said recessed area.

2. The measurement system of claim 1 wherein said mating relationship comprises a serpentine or zig-zag configuration.

3. The measurement system of claim 1 wherein a gap between said electrodes comprises a distance of from about 0.005 to 0.030 inches.

4. The measurement system of claim 1 wherein upon placement of the distal end of the housing on a mucosal surface of a patient an isolation chamber is formed that creates a microenvironment for the measurement of CO.sub.2.

5. The measurement system of claim 1 wherein the microenvironment prevents the dissipation of CO.sub.2. into surrounding areas.

6. The measurement system of claim 1 wherein said recognition layer is selected from a fluoropolymer, a polydimethylsiloxane, a hydrogel, a plastic material, an elastomeric material and combinations of the foregoing.

7. The measurement system of claim 1 wherein said recognition layer comprises a microporous membrane.

8. The measurement system of claim 1 wherein the recognition layer comprises a monolithic structure or a composite structure.

9. The measurement system of claim 7 wherein the recognition layer comprises a composite structure and the microporous membrane is operably coupled to one or both sides of the recognition layer.

10. The measurement system of claim 1 wherein said recessed area of said housing and said recognition layer form a covered, isolated microenvironment for the measurement of said carbon dioxide when placed on the mucosal surface of the patient such that said carbon dioxide does not dissipate from the microenvironment.

11. A method of measuring carbon dioxide in a tissue sample comprising:
providing a carbon dioxide measurement device comprising a housing having a distal end and a proximal end, said distal end including a recessed area; the housing including a plurality of electrodes positioned within said housing, said electrodes in mating relationship; a conductive media in contact with said electrodes; and a recognition layer disposed between said electrodes and said recessed area;
placing said carbon dioxide measurement device on a mucosal surface of a patient;
forming a microenvironment for the measurement of carbon dioxide on said mucosal surface; and
taking a carbon dioxide measurement of the patient.

12. The method of claim 10 wherein said mating relationship comprises a serpentine or zig-zag configuration.

13. The method of claim 10 wherein said recessed area of said housing and said recognition layer form a covered, isolated microenvironment for the measurement of said carbon dioxide when placed on the mucosal surface of the patient such that said carbon dioxide does not dissipate from the microenvironment.
